(19) United States
(12) Patent Application Publication
(10) Pub. No.: US 2003/0082168 A1
(43) Pub. Date: May 1, 2003

(54) Compositions and methods for facilitating weight loss

(76) Inventor: Inna Yegorova, Northridge, CA (US)

Correspondence Address: Nancy Lord Johnson, Ltd., 1970 N. Leslie Rd. No. 204, Pahrump, NV, 89060, US

(21) Appl. No.: 10/027,827

(22) Filed: Oct. 22, 2001

(51) Int. Cl. A61K 38/47; A61K 31/198; A61K 31/202; A61K 35/78; A61K 31/045

(52) U.S. Cl. 424/94.61; 424/750; 424/758; 514/562; 424/729; 514/561; 514/560; 514/729

(57) ABSTRACT

Compositions and methods for facilitating weight loss by inhibiting carbohydrate absorption, enhancing lipolysis, and modulating the metabolism of glucose in a human. The compositions comprise wheat alpha amylase, conjugated linoleic acid, Momordica charantia, lipotrophic vitamins and green tea.

### FIELD OF THE INVENTION

The present invention relates to the administration of novel compositions and related methods using wheat alpha amylase, conjugated linoleic acid and Momordica charantia, lipotrophic vitamins and green tea to facilitating weight loss by inhibiting carbohydrate absorption, enhancing lipolysis, and normalizing the metabolism of glucose in a human.

### BACKGROUND OF THE INVENTION

Obesity is a serious heath problem both in the United States as well as world-wide. Results from the National Health and Nutrition Examination Survey III show that one in three Americans are at least twenty percent overweight. Kuczmarski et al., 272 JAMA 205-211 (1994). Other studies have shown that the prevalence of obesity increases threefold between the ages of 20 and 50, however, this varies for men and women. In particular, the weights of men appear to stabilize after age 50 and then begin to decline around age 60. Women, however, generally continue to gain weight until age 60, and it is not until after age 60 that their weight begins to decline. Kaplan and Sadock, SYNOPSIS OF PSYCHIATRY 731 (1998).

Obesity is a condition characterized by excessive accumulation of fat on the body. Obesity can be measured by either body weight or by body mass index (BMI). By convention, obesity is said to be present when body weight exceeds by 20 percent the weight listed in typical height-weight index tables. The other measurement of obesity, BMI, is the amount of fat present in the body and is considered a reliable indication of fatness in non-athletic adults. The BMI may be calculated by using the following formula: BMI equals [body weight in kg] divided by [height in meters]². In general, a normal BMI is between the range of 20 to 25, whereas the BMI of obese individuals is greater than or equal to 30.

Individuals accumulate fat by eating more calories than are expended as energy. In other words, the intake of energy exceeds its dissipation. Indeed, if fat is to be removed from the body, fewer calories must be consumed or more calories must be expended than are put in. Specifically, the energy content of food is calculated from the heat released by the total combustion of food, which is expressed in kilocalories (Kcal or C). Part of the chemical energy released by the oxidation of fuel molecules is dissipated as heat, which in part is used to maintain body temperature. Approximately, 40% of the energy in food is captured in the synthesis of ATP (adenosine triphosphate) from ADP (adenosine diphosphate) and P_{I} (phosphate). Champe and Harvey, LIPPINCOTT's BIOCHEMISTRY REVIEWS 298 (1987).

The recommended daily allowance (RDA) for the minimal energy required for an individual has been approximated. Assuming light activity levels typical for most Americans, the recommended dietary energy intake for a 70-kg adult man is approximately 2900 Kcal. For a 50-kg adult woman it is about 2100 Kcal. The total energy that is required by an individual is the sum of three energy requiring processes that occur in the body. These include basal metabolism, specific dynamic action, and physical activity. Id., 298-299.

Basal metabolic rate (BMR) is the energy expended by an individual in a resting, postabsorptive state and represents the energy required to carry out the normal body functions. These include respiration, blood flow, and maintenance of neuromuscular integrity. In an adult, the BMR for men is roughly 1800 Kcal and 1300 Kcal for women. Approximately, 50% to 70% of the daily energy expenditure in sedentary individuals is attributable to the BMR.

Specific dynamic action, also referred to diet-induced thermogenesis, is related to the amount of heat produced by the body during the digestion and absorption of food. Specifically, the body heat production of the body increases as much as 30% above the basal level. Over a 24 hour period, the thermogenic response to food may amount to 5% to 10% of the total energy expenditure. Id., 299. In particular, relatively lean individuals may generate up to 40% above the basal level, while obese individuals barely generate a 10% increase above basal level. Murray and Pizzorno, ENCYCLOPEDIA OF NATURAL MEDICINE 684 (1998).

The final energy requiring process is physical activity. Indeed, it is physical activity that provides the greatest variation in energy expenditure. The amount of energy consumed during physical activity depends on the time and intensity of the exercise. In general, a sedentary person requires 30% to 50% more than the basal caloric requirement for energy balance, whereas a highly active individual may require 100% or more calories above BMR. Champe et al., supra.

A marked decrease in physical activity seems to be a major factor in the rise of obesity as a public health problem. Physical inactivity restricts energy expenditure and may contribute to increased food intake. Although food intake usually increases with increased physical activity, food intake does not necessarily decrease proportionately when physical activity falls below a minimum level. Id. at 735.

The best way to induce weight loss is through dieting or caloric restriction, i.e., establish a caloric deficit by bringing intake below output. The most common method of reducing caloric intake is by means of a low calorie diet, but there are several common pitfalls to dieting. One is that when the dieter stops the diet and returns to the usual fare, the incentives to overeat are multiplied. Second, many obese people choose novel or bizarre diets. One example of this type of diet is a high-fat/high-protein diet. These diets are known as ketogenic diets because they contain a high level of cholesterol, which facilitates ketosis. Ketosis is associated with nausea, hypotension, and lethargy. Kaplan, et al., supra, 735.

The allocation of energy utilization has some very interesting effects. For example, when an obese individual is subjected to food restriction, the body's reaction is to dramatically reduce basal metabolism. Thus, the weight loss from diet restriction is always less than the loss predicted from the caloric deficit. As the body gains weight, both the number of adipocytes and their lipid content increases. During food restriction, basal metabolism declines, and the lipid content, but not cell numbers of adipocytes decrease.decreases but the number of adipocytes remains constant. When caloric load is resumed, most of the food energy is directed into lipogenesis (to restore the obese weight set point), and there is a great excess of lipid depleted lipocytes available to absorb the new lipids. This explains why dieters tend to gain weight rapidly after discontinuing a fat-restricted diet.

This phenomenon is addressed by the PFL Consulting Company of York, Pa. in its popular diet product, Calorad.RTM. Calorad.RTM is a collagen hydrosolate administered at bedtime. It is said to enhance weight loss because up to 30% of the weight lost on a calorie-restricted diet may consist of lean muscle. Lean muscle loss adversely impacts weight loss by slowing the dieter's metabolic rate because one pound lean muscle burns 50 calories per hour while fat burns no calories. By supplementing collagen, Calorad.RTM is believed to maintain lean muscle, which in turn facilitates “fat” loss, while helping to maintain a healthy metabolic rate. Collagen hydrosylate is also found in Slender Now.RTM a weight loss kit that includes a starch blocker, thermogenic herbal formula, collagen hydrosylate, and low calorie drink, distributed by Life Plus Associates of Batesville, Ark.

Numerous other weight control products are known in the literature. One example of a weight control product is taught in U.S. Pat. No. 4,959,227 to Amer wherein the product has a reduced lactose content and contains dietary fiber. In similar fashion, U.S. Pat. No. 5,104,676 to Mahmoud et al. discloses a weight loss product that utilizes a particular blend of soluble, insoluble, fermentable and non-fermentable fibers. U.S. Pat. No. 6,113,949 to Brink, discloses a weight control product comprising a mixture of guggul extract and phosphate salts to enhance mood states, increase vigor and reduce blood serum lipid levels. U.S. Pat. No. 5,643,874 to Bremer, discloses a method for treatingobesity by administering a inhibitors of lipase, glucosidase, and amylase either simultaneously or in a chronologically spaced manner. Other commercially available products include Ultra-Slim Fast.RTM. which is distributed by Slim Fast Foods, a division of Thompson Medical Company, Inc.of New York, N.Y. and OpitiTrim.RTM. which is available from the Clinical Products Division of Sandoz Nutrition Corp.,Corp. of Minneapolis, Minn.

Acarbose is a complex oligosaccharide that competitively and reversibly inhibits alpha-glucosidase. Acarbose did not alter the emptying of a carbohydrate-free meal, but it delayed emptying of a mixed meal and a carbohydrate-free meal given 2 h after sucrose ingestion. Enc, F.Y., et al., 2001 AM. J. PHYSIOL.—GASTROINTEST.LIVER PHYSIOL. 281(3): G752-G763.

Surgical methods that cause malabsorption of food or reduce gastric volume have been widely used in people who are markedly obese. Gastric bypass is a procedure used to make the stomach smaller. In particular, the stomach is reduced by stapling or transecting one of the curvatures of the stomach. The primary objective of reducing the volume of the stomach is to slow the passage of food, thereby limiting the amount of food the individual can intake. Although the results are successful, many adverse side effects result. For example, vomiting, electrolyte imbalance, and obstructions may occur. An alternate surgical procedure is lipectomy, which is the removal of fat. This technique is primarily used for cosmetic purposes, however, and has no real impact on weight loss in the long run. Kaplan et al., supra, 276.

In addition, literally hundreds of chemical entities have been suggested as weight loss products. Hence, for all of the reasons detailed above, a better method for facilitating weight loss in obese or overweight individuals that is readily available and cost-efficient is needed. None of the prior art has suggested or disclosed the combined use of a wheat alpha amylase, conjugated linoleic acid, and Momordica charantia.

Therefore, the primary object of the present invention is the administration of a composition that is highly effective in reducing the weight of a human by simultaneously inhibiting the absorption of carbohydrates, enhancing lipolysis, and modulating sugar metabolism.

### SUMMARY OF THE INVENTION

One of the great challenges in modern medicine is to devise a safe and effective method of treating obesity and/or achieving healthy reductions in body weight to a predetermined level ideal for the individual. It is therefore an object of the present invention to provide a safe method of reducing weight and treating obesity. A second serious problem with obesity is that even when excess weight is lost, the effect is only temporary, and the individual regains weight soon after abandoning a diet or other treatment method. Heretofore, only lifelong adherence to a carefully formulated diet, and a program of regular exercise has succeeded in sustaining long term results. Accordingly, it is a second object of the invention to provide a lasting remedy for obesity without unpleasant restrictions in diet, or difficult to maintain exercise programs.

The present invention relates to compositions and methods for facilitating weight loss, inhibiting carbohydrate absorption, enhancing lipolysis, and modulating the metabolism of glucose in a human. The compositions comprise wheat alpha amylase, conjugated linoleic acid, and Momordica charantia. Preferably, the compositions also comprise lipotrophic vitamins choline, L-methionine and inositol, and a thermogenic substance, namely green tea.

In one preferred embodiment of the present invention, the composition may comprise wheat extract alpha that contains 50% alpha amylase inhibitor by weight, conjugated linoleic acid, and Momordica charantia in an extract that contains 10% bitter principle, choline bitartrate, L-methionine, inositol, and green tea present in an extract that contains 50% polyphenols

A further preferred embodiment of the present invention is a composition comprising between 8 mg and 200 mg wheat alpha amylase, between 60 mg and 1500 mg conjugated linoleic acid, between 24 mg and 600 mg Momordica charantia, between 16 mg and 400 mg choline, between 8 mg and 200 mg L-methionine, between 6 mg and 150 mg inositol and between 16 and 400 mg green tea. More specifically, the composition may comprise about 40 mg wheat alpha amylase, about 300 mg conjugated linoleic acid, about 120 mg Momordica charantia, about 80 mg choline, about 40 mg L-methionine, about 30 mg inositol and about 80 mg green tea.

In another embodiment of the present invention, the composition may be administered to a human. Administration may be oral, liposomal, inhalation, sublingual, rectal suppositories, oral spray and dermal patch. The composition preferably may be administered orally, preferably two or three times daily with meals.

### DETAILED DESCRIPTION OF THE INVENTION

The present invention relates to the administration of compositions and methods of facilitating weight loss by inhibiting carbohydrate absorption, enhancing lipolysis, and modulating the metabolism of glucose in a human. The compositions comprise wheat alpha amylase, conjugated linoleic acid, Momordica charantia, lipotrophic vitamins and green tea, and optionally lipotrophic vitamins preferably choline bitartrate, L-methionine, and inositol, and green tea.

It is understood that the present invention is not limited to the particular methodology, protocols, and reagents, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include plural reference unless the context clearly dictates otherwise.

Unless defined otherwise, all technical and scientific terms used herein have the same meanings as commonly understood by one of ordinary skill in the art to which this invention belongs. Preferred methods, devices, and materials are described, although any methods and materials similar or equivalent to those described herein can be used in the practice or testing of the present invention. All references cited herein are incorporated by reference herein in their entirety.

Wheat extract alpha as described herein may be standardized to, but is not limited to an extract containing 50% alpha amylase inhibitor.

Momordica charantia as described herein will also be referred to as gourdin and bitter gourd. Momordica charantia may be standardized to, but is not limited to an extract containing 10% bitter principle.

Choline, as described herein, includes the various forms of choline available, including but not limited to, choline bitartrate, phosphatidyl choline, lecithin, choline chloride, and cytidinediphosphocholine.

A patient, as described herein, includes individuals who require due to a disease state, treatment regimen, orneeddue to a disease state, treatment regimen, or desire, to reduce body weight or modulate glucose metabolism.

One aspect of the present invention is to inhibit the absorption of carbohydrate while enhancing lipolysis and modulating glucose metabolism to effect synergism between the several components of the composition. The addition of lipotrophic vitamins choline, L-methionine, and inositol, with or without green tea, further increases the effectiveness or the composition in facilitating weight loss.

In the preferred embodiment of the present invention, it is anticipated that there will be some variation in effectiveness because of differences among individuals in parameters such as body weight, basal metabolism, exercise, and other aspects of the diet. The individual should begin with the preferred dose of 3 capsules per day for an initial two week period, and then, if no weight loss is experienced, gradually increase the up to about 6 capsules per day.

Wheat Alpha Amylase

An amylase inhibitor of wheat origin is known to inhibit the activity of human pancreatic alpha-amylase and moderate the digestion of the starch ingested. It thus inhibits an increase in blood glucose level and reduces insulin secretion, so that the amylase inhibitor is effective for people having a high blood glucose level or are a candidate at risk for diabetes. It is also known that the amylase inhibitor of wheat origin is effective in weight control. Wheat amylase inhibitors may also be used to directly inhibit the accumulation of visceral fat as taught by Miyazaki, U.S. Pat. No. 5,789,380.

The rate of absorption of food is affected by various factors. Different carbohydrates (CHOs) containing equal amounts of CHO will induce different increases in postprandial glucose increments. They glycemic index is defined as the percentage of rise in blood glucose (area under the curve) after administration of CHO-CHO-containing food, divided by the rise of blood glucose after an equivalent amount of CHO in the form of glucose. Gastric emptying may be a factor responsible for the velocity of blood glucose increments after a meal, and fat slows gastric emptying and reduces the speed at which CHOs appear in the blood. Dreher, M L, et al 20(1) CRIT. REV. FOOD. SCI. NUTR. 47-71 (1984), 47 [Hereinafter “Dreher”].

Starch is composed of amylose and amylopectin. Amylose is composed of a large number of chains containing 16 to 28 glucose units linked by 1,4-alpha-glucopyranosidic bonds with a small percentage of interconnecting 1,6-alpha-glucopyranosidic branching units. Id, at 47. Starch is digested by monogastric animals first by the action of salivary and pancreatic alpha amylase to produce mainly maltose and alpha-limit dextrins (amylolysis), and second by the action of intestinal brush border glucosidases to produce glucose. Ibid, at 48.

Starch digestion is initiated in the mouth by the action of salivary alpha amylase, but this action is limited because starch remains in the oral cavity only a short time prior to amylase inactivation by the low pH of the stomach. Consequently, most starch is digested in the small intestine where pancreatic alpha amylase acts on starch in the lumen and along the fibers of the microvilli to release the end products composed largely of maltose, alpha-limit dextrins, and small amounts of glucose and maltotriose. These products are further digested to glucose as they come in contact with the intestinal surface membrane where brush-border enzymes act as the lumen-cell interface and the glucose is then actively transported across the mucosal membrane into the body. Usually, most starches are completely digested prior to reaching the ileum. Caspary W, 55 AM. J. CLIN. NUTR. 55: 299S-308S (1992).

Various non-degradable plant polysaccharides known as carbohydrate gelling agents such as guar and pectin inhibit the digestion and adsorption of carbohydrates. This is because there is an unstirred water layer covering the small intestinal mucosa that creates a luminal diffusion barrier. Substrates are taken up only when dissolved in the liquid medium whose thickness is correlated to absorption rates and the maximal transport rate. Reduced thickness accelerates absorption and rapid perfusion of the small intestine reduces the thickness of the unstirred layer and thus accelerates absorption. Guar seems to act by increasing thickness and reducing absorption rates. Id.

Generally absorption is the rate-limiting step, but for poorly digestible starch amylolysis may be rate limiting and since starch can be isolated from the colon, enterobacterial enzymes may also be involved in starch digestion. Dreher, supra, at 48. Starch that is undigested by the small intestine is fermented in the colon by anaerobic bacteria and metabolized to short chain fatty acids, hydrogen, carbon dioxide, and methane. Caspary, supra, at 303S. In this process, 27% of glucose carbohydrates are converted to metabolically inert products and lost as energy, while 62% is reused and effectively reabsorbed by the colon as short chain fatty acids by bacterial fermentation. Id.

On a nutritional level, amylase inhibitors appear to affect the rate of mammalian starch digestion, but this has been a controversial point since 1946 when Kneen and Sandstedt concluded that alpha amylase inhibitors from wheat are inactivated by pepsin and thus not of much nutritional significance. However, since that time some evidence has indicated that large amounts of the inhibitor may overcome gastric digestion in laboratory animals and man, but this has not been shown in several laboratory animal and most clinical studies. The most common type of amylase inhibitor is protein or glycoprotein in nature, alpha amylase-specific and relatively heat labile,labile and found in a wide variety of foods including wheat. Dreher, supra, at 61.

The ability of amylase inhibitor to reduce weight gain was demonstrated by several research groups in laboratory animals. Male weanling rats on a basal starch-containing diet showed 100% starch digestibility and an average daily weight gain of 1.52 g. The addition of amylase inhibitor up to 8% resulted in a reduction in starch digestion of up to 46%, shown by increased fecal starch, and an average daily weight gain to 0.85 g. Lang, J A, et al, 33 FED. PROC. 718 (1974). Marshal, reported unpublished work showing rats fed rat chow with autoclave-inactivated inhibitor grew at a 15 to 20% faster rate than did rats fed on chow containing the inhibitor. PHYSIOLOGICAL EFFECTS OF FOOD CARBOHYDRATES (A. Jeanes, A and J. Hodge, Eds. 1975, Ch. 15, p. 244). However, other studies in rats and chicks did not show a reduced growth rate with alpha amylase inhibitors. Dreher, supra, at 62.

In humans, Puls, W and Keup, N, 9 DIABETOLOGIA 97 (1973), measured the level of blood glucose and insulin after the ingestion of 100 g of raw and cooked starch with and without the inhibitor present. After ingestion of raw starch, the increases over basal levels of serum glucose and insulin were reduced in a dose-dependent fashion by the addition of the inhibitor. The effect was less marked with cooked starch, and required a larger dose. Raw potato and high-amylose starch gave low digestibility values of 78 and 66% respectively, and spray-dried wheat starch gave a value of only 76.8%. Cereal starches are generally more digestible than root/tuber and legume starches. Cooking improves digestibility of poor and intermediately digestible starches (%66). Fleming, S., 47 J. FOOD SCI. 1 (1982).

Amylase inhibitors enjoyed popularity in the early 80's when it was estimated that 10 million starch-blocker tablets were consumed weekly until the FDA intervened. Early studies were equivocal. In one such study, Garro, et al, 37C HUMAN NUTR. CLIN. NUTR. 301-305 (1983) five obese, non-diabetic, euthyroid women were given a tablet of one of two popular commercial starch blockers or placebo 30 minutes before eating. Breath carbon dioxide, a measurement of starch oxidation, and blood glucose were measured and no statistically significant differences were found between the tablets and placebo, leading the authors to conclude that the tablets exerted no measurable effect on digestion, absorption, or subsequent metabolism of starch.

Layer, et al, studied the effects of amylase inhibitors in humans. Layer, P., et al, 91(1) GASTROENTEROLOGY 41-8 (1986). Four fasting volunteers were intubated with an oroileal tube to obtain duodenal jejunal, and terminal ileal samples. After intubation, subjects ingested 50 g of rice starch given with placebo; on the second day; starch was given with 5-g or 10-g of the amylase inhibitor. Compared with placebo, the amylase inhibitor significantly (p less than 0.05) reduced duodenal, jejunal, and ileal intraluminal amylase activity by more than 95% for 1-2 h; increased postprandial delivery of total carbohydrate (glucose polymers in particular) to the distal small bowel;and reduced the early postprandial plasma glucose rise by 85%. The authors concluded that the more than 95% inhibition of amylase reduces dietary starch digestion within the small intestine and uptake of dietary starch from the small intestine, markedly decreases postprandial release of insulin and gastric inhibitory polypeptide, and may alter postprandial upper gastrointestinal motor function.

Without amylase inhibition, practically no starch reached the ileum. However when the amylase inhibitor was given, a large amount of carbohydrate passed the midjejunum and 20% was delivered to the terminal ileum and was associated with an increase in breath hydrogen. The breath hydrogen, along with absence of carbohydrate in stool sampled demonstrated that the colonic flora metabolized the malabsorbed carbohydrate. The authors concluded that amylase inhibition may be useful in treating obesity because the delay in gastric emptying increases feelings of satiety. However, the authors did not consider net loss of calories as a major consequence of amylase inhibition because the colonic flora efficiently metabolizes the malabsorbed carbohydrate, mostly to short-chain fatty acids.

Layer, P., et al, 88 GASTROENTEROLOGY 1895-902 (1985) also compared the effects of a commercial, bean-derived alpha-amylase inhibitor with a partially purified concentrated form of the inhibitor. Partially purified inhibitor into the human duodenum rapidly inhibited intraluminal amylase activity in a dose-dependent manner for>94% at 2.0 mg/ml to>99.9% at 5.0 mg/ml. A dose-dependent reduction in the rate of digestion of solid cooked and raw starch, but not liquid starch was noted, but this was considered of minimal importance because virtually all dietary starch is solid. The authors concluded that the failure of the commercial amylase inhibitor was due to insufficient anti-amylase activity.

On the basis of in vitro and early in vivo studies showing evidence that when ingested, phaseolamin, the alpha amylase inhibitor from kidney beans, could block the digestion of starch in meals, starch blockers increased from 3 in 1981 to over 300 in late 1982. The manufacturers of this product claimed that each tablet (containing approximately 8000 units of inhibitor activity) taken before or during a meal would block the absorption of several hundred calories of starch, enabling dieters to lose weight while they ate all the starch they wanted. By June 1982 the sale of starch blockers in the U.S. was about 10 million tablets per week. The relationship between starch blockers and ingested starch is clearly not as simple as the product manufacturers states, as Associated Press reported numerous cases of people taking the aid that required hospitalization due to nausea, vomiting, diarrhea, stomach pains, and excess gas. Bo-Linn, et al, 307 N. ENGL. J. MED. 1413 (1982), found that fecal calorie excretion was the same in subjects who had taken either a starch blocker or placebo after a high starch meal. Carlson, GL, 219(4583) SCIENCE 393-5 (1983), also found no significant effects in a randomized, double-blind, crossover clinical trial. One possible explanation for the failure of starch blockers to work is that starch is absorbed as volatile fatty acids following colon fermentation. Dreher, supra, at 63.

At that time, the Food, Drug and Cosmetic Act classified any substance that altered a body function as a drug and thus they so classified the starch blockers and required manufacturing and distribution to cease. Under the Dietary Supplement and Health Education Act of 1994, truthful structure function claims may be made for a dietary supplement, 21 U.S.C. 321 (r). This lead to further exploration of the potential for the use of starch blockers.

A Mayo Clinic study demonstrated that an alpha-amylase inhibitor isolated from beans effectively reduced postprandial blood glucose increments. Boivin, M., et al, 94 GASTROENTEROLOGY 387-92 (1988). Boivin, et al. studied the effects of 4-6 g of an amylase inhibitor in 6 non-insulin dependent diabetics. Postprandial glucose response was significantly reduced by 34% after breakfast (P<0.006), 11% after lunch (p=0.25) and 25% after supper (p<0.03). Fecal weight and outputs of dry matter, lactic acid and nitrogen were significantly greater during the week when the inhibitor was administered. Mild diarrhea occurred during the first two days of treatment then subsided. Only slight increases in fecal short chain fatty acids were observed, leading the authors to postulate that carbohydrate malabsorption results in more efficient digestion of the carbohydrate by colonic flora. Id. In an earlier study, subjects were randomized to receive either 2.0 or 2.9 gm of amylase inhibitor purified from Great Northern beans while ingesting a 650 calorie meal that consisted of 73.1 grams of carbohydrate. Each subject was tested with placebo seven days before the amylase test. Carbohydrate absorption was measured by breath H.Sub2 and plasma glucose. In those subjects who received 2.9 grams of the inhibitor, a mean of 18% of the ingested carbohydrate was malabsorbed. Boivin, M., 62 MAYO CLIN. PROC. 249-255, 250-1 (1987).

Amylase inhibitors of wheat origin may be made by any industrially efficient process for the preparation of amylase inhibitors having a high amylase inhibitory activity from the extract of wheat flour or the like by methods known to those of ordinary skill in the art.

Further examples of these are disclosed in Japanese Patent Kokai 5-301898, 7-48268 and 7-48400, Miyazaki, U.S. Pat. No. 5,789,380. Miyazaki discloses the method of Japanese Patent Kokai 5-301898 whereby the extract is obtained by (1) extracting wheat, wheat flour or wheat gluten with water, a dilute acid, a dilute alkali or an aqueous alcohol to obtain a solution containing an amylase inhibitor; (2) adding a polysaccharide to said solution to form an insoluble complex of the amylase inhibitor with the polysaccharide and separating the insoluble complex from the solution; (3) separating the polysaccharide from the complex separated in the above step to collect a solution containing the amylase inhibitor; and (4) treating the collected solution with a cation exchanger to recover the amylase inhibitor from the fractions that have not been adsorbedabsorbed on the cation exchanger.

Conjugated Linoleic Acid

Conjugated linoleic acid, referred to as “CLA” is a mixture of one or all of the isomers of octadecadienoic acid including the cis-9, trans-11; cis-9, cis-11; trans-9, cis-11; trans-9, trans-11; cis-10-cis-12; cis-10, trans-12; trans-10, cis-12; and trans-10, trans-12 cis-9, trans-11 and trans-10, cis-12 isomers are thought to have the most biological activity.

Studies in animals suggest that CLA enhances lipolysis, the breakdown of fat. In mice fed high-fat (45 Kcal %) or low-fat (15 Kcal %) diets with or without CLA (2.46 mg/Kcal; 1.2 and 1.0% by weight in high- and low-fat diets respectively) for 6 weeks. CLA significantly reduced energy intake, growth rate, adipose depot weight and carcass lipid and content independent of diet composition. CLA significantly increased metabolic rate and decreased nighttime respiratory quotient. After 6 weeks, the high-fat control group weighed 5.7 g more than the high-fat CLA group (P<0.0001) and the low-fat controls weighed 3.2 g more than the low-fat CLA group (P<0.005). During the sixth week, CLA increased energy expenditure suggesting that it was causally related to the loss of body fat. The mechanism is not fully understood, but the authors cite a report that suggests CLA directly stimulates lipolysis and decreases lipoprotein lipase. West, D., et al, 275 AM. J. PHYSIOL. (44 REGULATORY INTEGRATIVE COMP.PHYSIOL.) R667-R672 (1998), citing Park, Y., 32 LIPIDs 853-858 (1997)

Similar results were reported in pigs fed a cereal-based diet containing either 2% CLA or 2% sunflower oil. The CLA fed pigs tended to have reduced feed intakes, improved feed conversion efficiencies and similar rates of gain but less subcutaneous fat (−6.8%, P=0.01) and more lean muscle mass. The authors concluded that feeding CLA to pigs repartitions nutrients from fat toward lean deposition and tends to enhance feed conversion efficiency. Dugan, M., et al, CANADIAN J. ANIMAL SCI. 723-5

CLA is orally administered as a safe and effective method to decrease body weight in obese and non-obese humans. Because CLA is a non-toxic, naturally occurring food ingredient and not a drug, CLA may be consumed as a part of a normal diet and finds use as a part of everyday nutrition in people without obesity. U.S. Pat. No. 6,034,132 to Remmereit. Remmereit teaches the use of dosages of about 0.1 to 15 grams, most preferably 1.8 grams of CLA as therapeutically effective for treating clinically obese persons.

Cook, U.S. Pat. No. 5,814,663, teaches a method of treating a human to maintain a level of body fat at an existing level. Cook's method can be used by ex-dieters to maintain the lower level of body fat and/or body weight they have achieved or by persons who wish to increase their fat intake without increasing their level of body fat and/or body weight.

The mechanism by which CLA mediates these effects is not known, although some biochemical models involving fat partitioning and shifts in fatty acid precursor mediated synthesis of end product prostaglandins and leukotrienes have been proposed. It is known that CLA is taken up in triglycerides and phospholipids, and deposited in fat stores. The precise structure and distribution of these lipids is not known. Nor is it known whether there is a competitive incorporation amongst two or more isomers, or a preferential deposition of certain isomers in some lipid species over others. U.S. Pat. No. 6,034,132, to Remmereit.

Momordica charantia

Obese persons tend to remain so because their metabolism diverts from that of a lean person in several aspects. Obese persons had higher than normal glucose levels for 3 hours after breakfast, 2 hours after lunch, and 2 hours after dinner with fasting plasma insulin and plasma insulin levels at other sampling times were two to four times higher than in normal persons. These results were statistically significant. The insulin hyper-response was associated with small but significant decreases below fasting glucose levels. Genuth, S., 79 ANNALS INT. MED. 812-822 (1973).

In a second group of massively obese persons with abnormal carbohydrate toleranttolerance had higher plasma glucose levels than the first group and elevated plasma insulin levels. But in contrast to the obese with normal carbohydrate tolerance, mean peak insulin response was slower. Id. Two thirds of diabetic patients between ages 30 and 65 are obese. Obese overt diabetics have significantly elevated insulin levels over normal weight overt diabetics. Boshell, B., et al, 21 (12) AM. J. CLIN. NUT. 1419-1428 (1968)

Obesity is characterized by high levels of circulating insulin and defects in insulin receptors, with resistance to insulin's effects. Circulating lymphocytes from obese patients have show less binding of insulin than normals at the same insulin levels. Caloric restriction and weight loss produces improvements in binding of insulin to lymphocytes. Obese cells need more insulin in the medium to bind as many molecules of insulin, about twice the insulin concentration in the medium for equivalent binding to cells from thin subjects. Archer, J., Gorden, P., and Roth, J., 55 J. CLIN. INVEST 166-174, 168-9 (1975). After submission of their article, the authors discovered that the monocyte accounts for most insulin binding. They did not detect any systematic alteration in monocyte contents among cells from normals, obese patients, or insulin resistant females, but the same “negative cooperativity” as for lymphocytes was observed. Id.

This inventor has discovered that the addition of Momordica charantia to this composition helps to compensate for the differences in glucose metabolism and increased insulin resistance in the obese.

Endogenous glucose production is important for providing fuel to glucose-requiring tissues during fasting. Total glucose production consists of hepatic glycogenolysis and both hepatic and renal gluconeogenesis. During early fasting, total glucose production decreases because of a marked decline in glycogenolysis without a compensatory increase in the rate of gluconeogenesis. Obese subjects show a blunted decline in glucose production during fasting that may have been related to obesity-associated insulin resistance.

The normal decline in whole-body glucose production, whole-body glucose uptake, and adipose tissue glucose uptake during early fasting is attenuated in women with abdominal obesity. The alterations in glucose metabolism may be responsible for the blunted decline in plasma insulin and leptin concentrations during fasting, which may explain some of the differences in the metabolic response to fasting observed between lean and obese persons Horowitz, J., Coppack, S W, and Klein, S, 73(3) AMERICAN JOURNAl OF CLINICAL NUTRITION, 517-522, (2001).

The compositions of the present invention comprise Momordica charantia (gourdin). The most common preparations of gourdin make use of the fruit, the bitter gourd, or the seed. Its constituents include the Momordica p-fraction. Ng, T., et al, 15(1-2) AM. J. CHINESE MED. 31-42, 36, 39 (1987), that includes various saponins known as Momordicins.

Momordicin Ic is one of the active ingredients of gourdin and studies have demonstrated that it is present in clinically tested.effective. Momordica charantia is used here to improve the utilization of sugar and balance its metabolism.

In the tropics, fruits of Momordica charantia (bitter gourd) have been successfully used by diabetic patients. One of the first reports was in 1960, but animal models showed conflicting results. Raman, A, and Lau, C, 2(4) PHYTOMEDICINE 349-362, 351 (1996). Early researchers identified a substance in the fruit, polypeptide-z that resembled bovine insulin and produced large and statistically significant reductions in blood sugar level in maturity onset diabetics. Khanna, P, and Jain, S, 44(6) J. NAT. PROD. 648-655 (1981). This is an 11,000 Dalton protein. Ng, T., et al, 15(1-2) AM. J. CHINESE MED 31-42, 36, 39 (1987).

Ng T., et al, Id. were the first to examine the of insulin like activities such as inhibition of lipolysis and stimulation of lipogenesis in vitro. The seed “p-fraction” but not the fruit “p-fraction” had potent antilipolytic activity in rat adipocytes and inhibited the lipolysis induced by epinephrine, ACTH, Glucagon and dibutryl cyclic AMP. However, the seed fraction also inhibited lipogenesis determined by the incorporation of labeled glucose into lipid. Subsequently, inhibition of glucose absorption, insulin secretagogue activity and insulinomimetic effects were attributed to Momordica in vitro, but not all were supported in vivo.

The Momordica p-fraction can be isolated from Momordica charantia by methods well-known to those of ordinary skill of the art; for example the process described by Ng, T., et al. In diabetic subjects, Momordica charantia extract drink while fasting was shown to decrease serum glucose levels in both the fasting and postprandial states (2 hours after 75 gm oral drink) in 86% of 100 cases studied. Ahmad, N, 1999 BANGLADESH MED RES. COUNC. BULL. 25(l):11-13, 12. However, some authors however, report that Momordicins exhibit their hypoglycemic activity by suppressing the transfer of glucose from the stomach to the small intestine and by inhibiting glucose transport at the brush border of the small intestine. Matsuda H, et al., 46(9) CHEM. PHARM. BULL. 1399-403 (1998).

Monascus purpureus is available commercially around the world, through distributors such as Dr. Winfried Behr at Friedrich-Breuer-Str. 86-D-53225 Bonn, Allok at Lachenmeyrs TR. 18a, 81827, Munchen, Germany and Samlong Chemical Co., Ltd., P.B. Box 65, Changzhou, Jiangsu, China.

Liu, in U.S. Pat. No. 4,985,248, discloses a method for extracting the active portion of Momordica charantia. The fruit of Momordica charantia L. is dried and powdered. 5 liter of 90% ethanol is added to 1 kg of dried powder and the pH of the solution is adjusted to 2.5 with HCl. The solution is stirred and centrifuged and the supernatant saved. SnCl.sub.2 (50%) is added to supernatant and the pH is adjusted to 6.8 with NH.sub.4 OH. The solution is again centrifugescentrifuged and the precipitate saved dissolved in acid solution. A solution of NaCl is added to the acid solution and a precipitate formed. The precipitate is then washed with acetone and vacuum dried.

Lipotrophic Vitamins

Lipotropics can increase the mobilization of fatty acids, thus helping people to lose body fat. These include choline, methionine, folate and Vitamin B₁₂ are important for lipid metabolism and synthesis of cellular membranes. Deficiency leads to lipid in liver and later to cirrhosis and can result in liver cancer. Newberne, PH, 206 ADV. EXP. MED. BIOL. 223-51 (1986). Sixty years ago, it was discovered that inositol prevented biotin induced fatty liver in rats, resembling lipocaic, which prevents the acutely fatty livers of rats caused by feeding beef liver fractions. Gavin, G, and McHenry, E, 139 J. Biol. Chem. 485 (1941).

Insulin was discovered in 1921 by Banting at the University of Toronto. Banting extracted pancreatic fluid by tying off the pancreatic ducts of a dog and allowing it to accumulate. He then injected it into a depancreatized dog and within an hour the dog's blood sugar level dropped 40% and his condition improved remarkably. In 1922, a 14-year-old boy was the diabetic human to receive insulin. Newberne, supra, at 224. However, it was discovered in subsequent years that the depancreatized dog receiving insulin failed to survive more than 11 months on a diet of lean meantmeat and sugar, and autopsy revealed extensive fat infiltration of the liver. The addition of raw pancreas to the diet prevented the condition.

These observations led to the discovery that 10 grams of lecithin daily in the diet restored the animals to normal, the same as raw pancreas. Researchers then demonstrated the relationship between choline deprivation and liver damage in a large number of species. Lipotrope deficiency decreases DNA methylation and S-adenyl methionine (SAM) and the DNA has less S-methyl cytosine, a substance that turns off gene expression and protects against differentiation into neoplasm. Newberne, supra, at 234. Choline inhibits the accumulation of triacyl-glycerols and promotes the removal of excess fat from the liver.

However, lipotrope deficiency is not the same as choline deficiency, though both can increase triacyl-glycerols and lead to fatty liver and lipodiastemata, a condition characterized by the fusion of neighboring hepatocytes. Lipotrope deficiency syndrome is prevented by the addition of adequate choline, methionine, folic acid, Vitamin B₁₂. A deficiency of either choline or lipotropes leads to a decrease in phosphatidyl choline and a decrease in VLDL, and choline deficiency leads to an increase in free radicals. Ghoshal, A, and Farber, E, 68(3) Lab. Invest. 255-260 (1993).

The compositions of the present invention preferably comprise choline, preferably as choline bitartrate, and L-methionine, and inositol. These lipotrophic vitamins are administered to in an appropriate dosage form to facilitate weight loss with wheat extract alpha and Momordica charantia.

Green Tea

Another aspect of the present invention is to regulate or enhance metabolism. This can be accomplished by administering a composition containing thermogenic substances, which will raise an individual's metabolic rate, and thus encourage weight loss. Use of thermogenic agents, especially when an individual is dieting, are of great importance. Caloric restriction during dieting causes the body's metabolic rate to slow. It is this slowing metabolism that promotes the storage of fats. By utilizing a thermogenic enhancing agent, the body maintains a high metabolic rate, which causes the body to burn the caloric intake as energy as opposed to promoting it to be stored as fat.

It has long been recognized that flavonoids inhibit the enzyme catechol O-methyltransferase. Borchardt, R, and Huber, J, 18(1) Jl. Med. Chem. 120-122 (1975); Green tea contains flavonoids, catechins polyphenols and caffeine. To treat obesity, there are two fundamental methods, reduce energy intake or increase energy expenditure (EE). Thermogenesis and fat oxidation are largely controlled by the sympathetic nervous system. The concentration of norepinephrine at the synaptic junction and its interaction with adrenoceptors is likely to by reduced by enzymatic degradation by catechol O-methyltransferase (COMT). Dulloo, A, 70 AM. J. CLIN. NUTR. 1040-5, 1040 (1999). Id, at 1040. Caffeine is one of the most widely used thermogenic agents.

There is evidence that the enzyme catechol O-methyltransferase (COMT) can be inhibited by tea polyphenols. Id. The predominant catechin polyphenol found in green tea is (−)-epigallocatechin gallate (70%). In vitro measurements of oxygen uptake of intercapsular brown adipose tissue (IBAT) of recently killed rats were compared between suspensions of IBAT in green tea, caffeine, green tea and ephedrine, and ephedrine and caffeine. Caffeine alone does not increase IBAT oxygen uptake, but green tea containing isomolar concentrations of caffeine resulted in significant, dose-depending increases in IBAT oxygen uptake to a 5-fold increase at 250 micro moles (UM). The addition of ephedrine produced a synergistic effect in increase of oxygen uptake, but it was stronger with green tea than with caffeine. Chemical sympathectomy blunted this effect, demonstrating that theenzyme is mediated through interference with noradrenaline (norepinephrine) released from sympathetic nerves. Dulloo, A., et al, 24 INT. J. OBESITY 252-258 (2000).

There are several studies that demonstrate that increasing energy expenditure though enhanced thermogenesis leads to significant weight loss. In vitro results showed that green tea extract was more effective than equivalent amounts of caffeine. The difference between the green tea extract and equimolar caffeine in activating thermogenesis was much more marked under conditions of increased norepinephrine release because of the synergistic interaction between green tea extract and ephedrine on tissue thermogenesis was more pronounced than that of caffeine or ephedrine. Dulloo, supra, at 1041.

Dulloo studied the energy expenditure (EE) in healthy young men treated with placebo, caffeine or green tea extract. Diurnal and 24 hour EE were significantly higher in the green tea group and urinary norepinephrine and its precursor dopamine tended to be highest during treatment with green tea, and the difference was statistically significant for 24-hour EE. Subjects were monitored in a respiratory chamber, a small room equipped to be airtight with differential analyzers for continuous monitoring of measurements in oxygen and carbon dioxide content to determine the respiratory quotient (RQ). The oxidation rates of protein, carbohydrate, and fat were calculated from the 24-hour EE, respiratory quotient, and urinary nitrogen excretion. Dulloo, supra, at 1042.

The differences between oxidation of substrates, in both grams and percent, were statistically significant as between the green tea group and both the placebo and caffeine groups. These values were lower for carbohydrate and higher for fat in the green tea group. The differences in substrate utilization in favor of fat oxidation in response to the green tea extract were consistently observed in most subjects. Dulloo, supra, at 1043. The authors suggest that the tea catechin polyphenols, by inhibiting COMT, increase or prolong the effect of norepinephrine on thermogenesis and fat metabolism or both.

The compositions of the present invention also preferably comprise green tea.

Formulation

According to one embodiment of the invention, the present composition is formulated for oral administration. Any dosage form may be employed for providing the patient with a dosage of the present compositions. Administration may be oral, liposomal, inhalation, sublingual, rectal suppositories, oral spray and dermal patch. Dosage forms include tablets, capsules, dispersions, suspensions, solutions, capsules, transdermal delivery systems, etc. Tablets and capsules represent the most advantageous oral dosage unit form. Any method known to those of ordinary skill in the art may be used to prepare capsules, tablets, or other dosage formulations. Tablets or capsules can be coated by methods well known to those of ordinary skill in the art.

According to one aspect of the invention a composition is provided comprising a pharmaceutically acceptable combination of the composition and at least one carrier. Pharmaceutically acceptable carriers for inclusion into the present compositions include carriers most suitable for combination with lipid-based drugs such as diluents, excipients and the like, which enhance its oral administration. Suitable carriers include, but are not limited to, sugars, starches, cellulose and derivatives thereof, wetting agents, lubricants such as sodium lauryl sulfate, stabilizers, tableting agents, anti-oxidants, preservatives, coloring agents and flavoring agents. Pharmaceutically acceptable carriers include binding agents such as pregelatinized maize starch, polyvinylpryrrolidone or hydroxypropyl methycellulose; binders or fillers such as lactose, pentosan, microcrystalline cellulose or calcium hydrogen phosphate; lubricants such as magnesium stearate, talc or silica; disintegrants such as potato starch or sodium starch; or wetting agents such as sodium lauryl sulfate. Reference may be made to REMINGTON's PHARMACEUTICAL SCIENCES, 17TH ED., 1985, for other carriers that would be suitable for combination with the present compositions. As will be appreciated, the pharmaceutical carriers used to prepare compositions in accordance with the present invention will depend on the administrable form to be used.

Another embodiment of the invention involves administering the composition of the present invention to a human in one or more tablets as a material dietary supplement. In yet another embodiment of the invention, the composition is administered to a human as a pharmaceutical composition. The administration of the composition is preferably in accordance with a predetermined regimen, which may be at least once daily and over an extended period of time as a chronic treatment, and could last for one year or more, including the life of the host. The dosage administered will depend upon administration frequency, the blood level of the components of the composition desired, other concurrent therapeutic treatments, the condition's severity, whether the treatment is for prophylaxis or therapy, the patient's age, the degree of weight loss desired, and the like.

All of the ingredients in the composition are available commercially, in bulk and wholesale, from suppliers well known to those with ordinary skill in the art. For instance, Wholesale Vitamins USA, Inc., of Brooklyn, N.Y. offers over 8,000 vitamins at wholesale prices

Other objects, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description.

### EXAMPLE 1

### Composition 1

A composition of the following formulation was prepared in tablet form by standard methods known to those skilled in the art:

| | |
|---|---|
| Wheat extract alpha (50% | 40 mg |
| amylase inhibitor | |
| Conjugated linoleic acid | 300 mg |
| Momordica charantia (10% | 120 mg |
| bitter principle | |
| Green tea (50% polyphenols | 80 mg |
| Choline bitartrate | 80 mg |
| L-Methionine | 40 mg |
| Inositol | 30 mg |

Three tablets per day is the recommended dosage for an average weight adult human (70-kg), taken with meals.

Clinical Studies

In the post-marketing surveillance of the invention, 300 customers were selected solely on the basis of the date of purchase. Fifty-seven individuals responded, and their comments are noted in Table 1.

TABLE 1

| | Num- | |
|---|---|---|
| Effect | ber | Comments |
| WEIGHT LOSS | 29 | Losing weight slowly but consistently; Alpha |
| | | Extract [AE] stopped the weight gain, so now |
| | | can maintain the same weight; fat deposits |
| | | burned; lost ½-1 lbs per week; lost 10 lbs |
| | | in 6 weeks; lost 8 lbs in a month; helped not to |
| | | gain weight over the holiday season; after only |
| | | a month of taking it, experienced less bloating |
| | | and a few inches came off the waist; lost 20 |
| | | lbs; lost 7 lbs in 2 months; lost 40 lbs in 4 |
| | | months; reversed a creeping weight gain over |
| | | past 6 months and lost 5 lbs; lost 5 lbs in 2 |
| | | weeks; lost 12 lbs in 6 weeks; AE not only |
| | | helps to lose weight (20 lbs), but also to keep |
| | | weight off; lost 7 lbs with very little effort |
| DIGESTION | 6 | Improved digestion; digestion seems better; not |
| | | as bloated |
| BLOOD SUGAR/ | 5 | Lower blood sugar; sugar level went down; |
| CHOLESTEROL | | stabilizes sugar levels; as a type II diabetic was |
| | | able to reduce insulin injections by 30%; |
| | | blood pressure is down |
| METABOLISM | 3 | Metabolism operated at enhanced, increased |
| | | levels; metabolism is up |
| APPETITE | 8 | Lost appetite for fatty snacks; no craving for |
| | | sweets; less appetite before the meals; not as |
| | | many food cravings; don't stuff self when eat; |
| | | do not feel hungry as often |
| ENERGY | 15 | Energy level seems higher; have a lot more |
| | | energy and feel much happier; do not feel as |
| | | tired as before; feel more like walking daily |
| SLEEP | 1 | Sleep better |
| NO EFFECT | 18 | |

Of 57 responders, 29 reported facilitated weight loss and 15 reported increased energy levels.

### EXAMPLE 2

A study of the effect of a dietary supplement comprising wheat extract alpha, conjugated linoleic acid, Momordica charantia, lipotrophic vitamins choline bitartrate, L-methionine, and inositol, and green tea on insulin levels, dietary intake, body mass index, and physical activity, is conducted over a six-month period. a statistical analysis is performed to compare the resulting insulin levels, dietary intake, body mass index, and physical activity of the test and a control (placebo) group to measure the improvement in insulin sensitivity, dietary intake, body mass index, and physical activity from administration of the test preparation.

Sixty men having a body mass index of greater than 30 are selected for inclusion in the statistical study. Two weeks prior to the start of the study, each subject completes a one-week dietary intake record and is interviewed by a Registered Dietitian to calculate each individual's daily energy requirement. After this, weight calculations, physical activity levels, and dietary intake levels are determined, and baseline blood samples are drawn on two separate days, then the subjects are randomly assigned to one of two treatment groups: the group receiving the test tablets (Composition 1) or the group receiving matching placebo tablets. Both groups continue on their normal daily diet and incorporate three tablets of either the Composition 1 or placebo in their daily diet. Each group documents their daily dietary intake, their level of physical activity, and their weight for the period of the study.

The effects of supplementing the diet with the above composition are evaluated for insulin sensitivity, dietary intake, body mass index, and physical activity using multiple linear regression analysis and a standard students t-test. In each analysis, the baseline value of the outcome variable is included in the model as a covariant. Treatment by covariant interaction effect is tested by the method outlined by Weigel and Narvaez, 12 CONTROLLED CLINICAL TRIALS 378-394 (1991). In the absence of significant interaction effects, the interaction terms are removed from the model. Regression model assumptions of normality and homogeneity residual variance are evaluated by inspection of plots of residuals versus plots of predicted values. Temporal outset of effects is detected sequentially by testing for the presence of significant treatment effects at 18, 12, and 6 weeks, proceeding to the earlier time in sequence only when significant effects have been identified at each later time period. In addition, one-way analysis of variance is compared only when differences exist between groups concerning nutrient intake, physical activity, and body mass index at each time point. Changes from the baseline within each group are evaluated using paired t-tests. Additionally, analysis of variance is performed on all baseline measurement and measurable subject characteristics to assess homogeneity between groups. All statistical procedures are conducted using the Statistical Analysis System (SAS Institute Inc., Cary N.C.) using an alpha level of 0.05 for all statistical tests.

A statistically significant increase in physical activity and a statistically significant decrease in dietary intake and body mass index are observed in the treated subjects upon completion of the study, but not in the control subjects. A statistically significant increase in insulin sensitivity is observed in the treated subjects upon completion of the study, but not in the control subjects.

The invention has been described in detail with particular reference to preferred embodiment thereof. However, it will be appreciated that those skilled in the art, upon consideration of this disclosure may make variations and modifications within the spirit and scope of the invention.

The disclosure of all publications cited above are expressly incorporated by reference in their entireties to the same extent as if each were incorporated by reference individually.

What is claimed is:

1. A composition for facilitating weight loss comprising the alpha-amylase inhibitor wheat alpha amylase, conjugated linoleic acid, and Momordica charantia.

2. The composition of claim 1 comprising between 8 mg and 200 mg wheat alpha amylase, between 60 mg and 1500 mg conjugated linoleic acid, and between 24 mg and 600 mg Momordica charantia.

3. The composition of claim 2 comprising about 40 mg wheat alpha amylase, about 300 mg conjugated linoleic acid, and about 120 mg Momordica charantia.

4. The composition of claim 1 further comprising one or more lipotrophic vitamins selected from the group of choline, lecithin, L-methionine and inositol.

5. The composition of claim 4 wherein said one or more lipotrophic vitamins are choline, L-methionine and inositol.

6. The composition of claim 5 wherein said lipotrophic vitamins are contained in the composition in amounts between 16 mg and 400 mg choline, between 8 mg and 200 mg L-methionine, and between 6 mg and 150 mg inositol.

7. The composition of claim 6 wherein said lipotrophic vitamins are contained in the composition in amounts of about 80 mg choline, about 40 mg L-methionine and about 30 mg inositol.

8. The composition of claim 1 further comprising a thermogenic substance, namely, green tea.

9. The composition of claim 8 wherein said green tea is contained in the composition in an amount between 16 mg and 400 mg.

10. The composition of claim 9 wherein said green tea is contained in the composition in an amount of about 80 mg.

11. The composition of claim 1, further comprising a pharmaceutically acceptable carrier.

12. The composition of claim 1, wherein said wheat extract alpha contains about 50% alpha amylase inhibitor by weight.

13. The composition of claim 1, wherein said Momordica charantia is present in an extract that contains about 10% bitter principle.

14. The composition of claim 4, wherein said choline is choline bitartrate.

15. The composition of claim 8, wherein said green tea is present in an extract that contains about 50% polyphenols.

16. A method of facilitating weight loss, inhibiting carbohydrate absorption, enhancing lipolysis, and normalizing the metabolism of glucose in a human comprising administering to said human a composition comprising wheat alpha amylase, conjugated linoleic acid, and Momordica charantia.

17. The method of claim 15 wherein said wheat extract alpha contains about 50% alpha amylase inhibitor by weight.

18. The method of claim 15 wherein said Momordica charantia is administered in an extract that comprises about 10% bitter principle.

19. The method of claim 16 wherein said composition comprises between 8 mg and 200 mg wheat alpha amylase, between 60 mg and 1500 mg conjugated linoleic acid, and between 24 mg and 600 mg Momordica charantia.

20. The method of claim 19 wherein said composition comprises about 40 mg wheat alpha amylase, about 300 mg conjugated linoleic acid, and about 120 mg Momordica charantia.

21. The method of claim 14 wherein said administration is selected from the group consisting of peroral, liposomal, inhalation, sublingual, rectal suppositories, oral spray and dermal patch.

22. The method of claim 14 wherein said composition further comprises one or more lipotrophic vitamins selected from the group consisting of choline, lecithin, L-methionine, and inositol.

23. The method of claim 19 wherein said lipotrophic vitamins are choline, L-methionine, and inositol.

24. The method of claim 20 wherein said lipotrophic vitamins are present in the composition in amounts between 16 mg and 400 mg choline, between 8 mg and 200 mg L-methionine, and between 6 mg and 150 mg inositol.

25. The method of claim 19 wherein said lipotrophic vitamins are present in the composition in amounts of about 80 mg choline, about 40 mg L-methionine, and about 30 mg inositol.

26. The method of claim 14 wherein said composition further comprises a thermogenic substance, namely green tea.

27. The method of claim 23 wherein said green tea is present in the composition in an amount between 16 mg and 400 mg.

28. The method of claim 24 wherein said green tea is present in an amount of about 80 mg.
